(19) United States
(12) Patent Application Publication
(10) Pub. No.: US 2015/0010918 A1
(43) Pub. Date: Jan. 8, 2015

(54) IMMUNOCHROMATOGRAPHIC ASSAY WITH MINIMAL REAGENT MANIPULATION

(71) Applicant: NOVAMED LTD., Jerusalem (IL)

(72) Inventor: Igor Ruvinsky, Ramat Gan (IL)

(21) Appl. No.: 14/371,499

(22) PCT Filed: Jan. 15, 2013
(86) PCT No.: PCT/IL2013/050042

§ 371 Date: Jul. 10, 2014

(30) Foreign Application Priority Data
Jan. 16, 2012 (IL) 217569

(51) Int. Cl. G01N 33/569

(52) U.S. Cl. CPC G01N 33/56944
USPC 435/7.1; 435/287.2

(57) ABSTRACT

The present invention provides a method for determining the presence or absence of an analyte in a sample with minimal sample preparation. The invention further provides immuno-chromatographic devices for determining the presence or absence of an analyte in a sample from which the analyte should be extracted, this device comprising an analyte extraction zone wherein one or more mobile extraction solutions are impregnated, and wherein at least one of said extraction solutions comprises an animal serum or plasma component.

### FIELD OF THE INVENTION

The invention relates to the field of medical diagnostic and devices for detecting analytes in biological samples. More particularly, the present invention relates to a rapid assay for the detection of Streptococcal antigens, wherein minimal reagent manipulation is required.

### BACKGROUND OF THE INVENTION

Among the beta-hemolytic streptococci causing infections in humans, the A, B, C and G groups figure most prominently⁵. Group A streptococci continue to be a focus of interest not only because of their causal role in acute streptococcal pharyngitis and other pyogenic infections but also because of their association with post streptococcal sequelae, specifically acute rheumatic fever and acute glomerulonephritis^{1,2}. In order to properly treat the disease using antibiotic therapy, it is important to use an accurate diagnostic method to identify the pathologic agent. For the screening of Group A streptococcal infection several methods are currently used including susceptibility of the organism to a bacitracin disc placed on a sheep blood agar plate, latex agglutination and enzyme immunoassay^{3,4}. More recently, rapid immunoassays have been developed for the diagnosis of Group A Streptococcus, such as immunochromatographic assays, and more particularly “lateral flow immunoassays”.

Group B streptococci are most notable for their role in causing neonatal sepsis and meningitis. Two forms of neonatal infection have been recognized on clinical and epidemiological grounds: (a) Early onset disease, which usually occurs within the first 10 days postpartum; (b) Late onset disease, which usually, though not always, occurs 10 days postnatally. Late onset disease may be due to nosocomial acquisition of the organism and is rarely associated with material or obstetric complications. Early onset disease is thought to be due to acquisition, perhaps by aspiration, of the organism from the mother's genital tract at the time of delivery. The incidence of Group B Streptococci ranges from 1 to 5% of live births, with mortality rates from 22 to 80%. In order to properly treat the disease using antibiotic therapy, it is important to use an accurate diagnostic method to identify the pathologic agent. Different methods are currently used for detection of Group B Streptococci including cellular culture, immunofluorescence, enzyme immunoassay, or latex agglutination.

Immunochromatographic assays are immunoassays enabling the user to add a sample to a sample receiving zone and obtain a positive or negative signal reporting the presence or absence of the analyte of interest in the sample. In lateral flow immunoassays, the sample is loaded in a sample receiving zone and allowed to flow laterally to a reaction zone situated on the surface of a porous membrane. A capture reagent is present in the reaction zone and the captured analyte is detected via a visible moiety or visible reaction associated with the capture reagent. Examples of such lateral flow immunoassays have been described for instance in U.S. Pat. Nos. 4,861,711, 4,943,522, U.S. Pat. No. 4,956,302, WO 92/12428, WO 97/06439.

Various lateral flow immunoassays for the diagnosis of Group A Streptococcus from throat swabs are commercially available. However such tests necessitate a preparation step which involves contacting the throat swab with several reagents. For instance, in Strep A immunoassays from Abbott, 3 drops of 2.0M sodium nitrite and 3 drops of 1.0M acetic acid are mixed prior to the extraction procedure, and 3 additional drops of 1.0M of Tris buffer is added before running the immunoassay. In the Applied Biotech, Inc. tests, extraction is made in a solution comprising 3 drops of 1.0M sodium nitrite and 3 drops of 1.25M acetic acid. The immunoassay is then performed after the neutralization of the extraction solution with 3 drops of 0.1M Tris-0.7M sodium hydroxide. The above-described preparation procedures, if not performed by a skilled worker, may lead to erroneous diagnosis. For instance, false negative results may easily arise if the Strep A antigen is not extracted properly from the throat swab, reducing the amount of antigen below the minimum detectable amount of the immunoassay.

Therefore, there is still the need to develop immunochromatographic assays for determining the presence or absence of Streptococcal antigens in a sample, especially Strep A and/or Strep B antigens, which do not require a complex extraction procedure of the antigens from the sample.

It is an object of the present invention to provide a method for detecting an analyte in a sample with a minimal number of sample preparation steps.

In particular, it is an object of the invention to provide a method for detecting Strep A and/or Strep B antigens in a sample wherein the extraction of the antigen is not performed by the user prior to testing.

It is another object of the invention to provide an immunochromatographic device for detecting an analyte in a sample, wherein the extraction of the analyte is performed within said device.

In particular, it is an object of the invention to provide an immunochromatographic device for the detection of any kind of Streptococcal antigens in a sample, wherein the extraction of said antigens is performed within said device.

Other features and advantages of the invention will be apparent from the following detailed description of the invention in conjunction with the accompanying drawings and from the claims.

### SUMMARY OF THE INVENTION

In a first aspect, the present invention provides a method for determining the presence or absence of an analyte in a sample with minimal sample preparation, the method comprising:
(a) providing a sample for which the presence or absence of an analyte should be determined and from which the analyte should be extracted;
(b) diluting the sample in a water-based solution to obtain a sample solution;
(c) transferring a volume of the sample solution to an immunochromatographic device comprising at least one porous element allowing the flowing of liquid solutions;
(d) extracting the analyte from the sample solution within the immunochromatographic device by contacting the sample solution with at least one mobile extraction solution impregnated in the device;
(e) forming a labeled analyte-binding agent complex by contacting the solution comprising the extracted analyte with a mobile solution comprising a labeled analyte-specific binding agent;
(f) determining the presence or absence of the analyte in the sample by the presence or absence of a signal formed by the binding of the labeled analyte-binding agent complex to an analyte capturing agent specific for the analyte or the labeled analyte-binding agent complex; and
(g) determining the presence of a positive control signal by the presence or absence of a signal formed by the binding of a control agent to a control capturing agent or the binding of the labeled analyte-specific binding agent of step (e) to a control capturing agent;
   the above method being characterized in that at least one of the extraction solutions of step (d) comprises an animal serum or plasma component.

In some embodiments of the method of the invention, the analyte is a Streptococcal antigen, and in specific cases a Group A Streptococcal antigen (Strep A) and/or a Group B Streptococcal antigen (Strep B).

In some embodiments of the method of the invention, the sample is a swab sample such as a throat swab sample or a bacteriological loop sample.

In some embodiments of the method of the invention, step (b) consists in diluting the sample in a water-based solution comprising an acid, and at least one of the extraction solutions of step (d) comprises a nitrite salt and an animal serum or plasma component. In some other embodiments, step (b) consists in diluting the sample in a water-based solution comprising a nitrite salt, and at least one of the extraction solutions of step (d) comprises an acid and an animal serum or plasma component. In still other embodiments, step (b) consists in diluting the sample in a water-based solution, and a first extraction solution of step (d) comprises an acid, a second extraction solution of step (d) comprises a nitrite salt, and either the first extraction solution, the second extraction solution, or both extraction solutions comprise an animal serum or plasma component. In some specific embodiments, the acid is selected from the group consisting of acetic acid, citric acid, sulfuric acid, hydrochloric acid, nitric acid, phosphoric acid and weak organic acids. The acid concentration in the water-based solution or the extraction solution is in a range selected from about 0.05 M to about 0.6 M, about 0.2 M to about 0.5 M, or about 0.35 M to about 0.45 M. In some specific embodiments, the nitrite salt is sodium nitrite. The concentration of nitrite salt in the water-based solution or the extraction solution is in a range selected from about 0.5 M to about 6 M, about 2 M to about 5 M, or about 3.5 M to about 4.5 M.

In some embodiments of the method of the invention, the animal serum or plasma component is selected from the group consisting of fetal bovine serum (FBS), fetal calf serum (FCS), and donor goat serum (DGS). The concentration of animal serum or plasma component is in a range selected from about 5% (v/v) to about 20% (v/v), about 7.5% (v/v) to about 15% (v/v), or about 9% (v/v) to about 11% (v/v).

In the method of the invention, no dedicated neutralization step is required further to extraction step (d).

In a particular embodiment of the method of the invention, the labeled analyte-specific binding agent of step (e) is a gold-labeled anti-Strep A mono- or polyclonal antibody; the analyte capturing agent of step (f) is an anti-Strep A monoclonal or polyclonal antibody; and the control capturing agent of step (g) is a mono- or polyclonal antibody raised against the species of the host animal in which the gold-labeled anti-Strep A antibody of step (e) was raised.

In another particular embodiment of the method of the invention, the labeled analyte-specific binding agent of step (e) is a gold-labeled anti-Strep B mono- or polyclonal antibody; the analyte capturing agent of step (f) is an anti-Strep B monoclonal or polyclonal antibody; and the control capturing agent of step (g) is a mono- or polyclonal antibody raised against the species of the host animal in which the gold-labeled anti-Strep B antibody of step (e) was raised.

In a further aspect, the present invention provides an immunochromatographic device for determining the presence or absence of an analyte in a sample from which the analyte should be extracted, this device comprising an analyte extraction zone wherein one or more mobile extraction solutions are impregnated, and wherein at least one of the extraction solutions comprises an animal serum or plasma component.

In some embodiments of the immunochromatographic device of the invention, the analyte is a Streptococcal antigen and in specific cases a Group A Streptococcal antigen (Strep A) and/or a Group B Streptococcal antigen (Strep B).

In some embodiments of the immunochromatographic device of the invention, the sample is a swab sample such as a throat swab sample or a bacteriological loop sample.

In some embodiments of the immunochromatographic device of the invention, one of the extraction solutions comprises a nitrite salt and an animal serum or plasma component. In some other embodiments of the immunochromatographic device of the invention, one of the extraction solutions comprises an acid and an animal serum or plasma component. In still other embodiments of the immunochromatographic device of the invention a first extraction solution comprises an acid and a second extraction solution comprises a nitrite salt, and either the first extraction solution, the second extraction solution, or both extraction solutions comprise an animal serum or plasma component. In some embodiments, the acid is selected from the group consisting of acetic acid, citric acid, sulfuric acid, hydrochloric acid, nitric acid, phosphoric acid and weak organic acids. The acid concentration in the extraction solution is in a range selected from about 0.05 M to about 0.6 M, about 0.2 M to about 0.5 M, or about 0.35 M to about 0.45 M. In some particular embodiments, the nitrite salt is sodium nitrite. The concentration of nitrite salt in the extraction solution is in a range selected from about 0.5 M to about 6 M, about 2 M to about 5 M, or about 3.5 M to about 4.5 M. In some particular embodiments of the device of the invention, the animal serum or plasma component is selected from the group consisting of fetal bovine serum (FBS), fetal calf serum (FCS) and donor goat serum (DGS). The concentration of animal serum or plasma component is in a range selected from about 5% (v/v) to about 20% (v/v), about 7.5% (v/v) to about 15% (v/v), or about 9% (v/v) to about 11% (v/v).

In a still further aspect, the invention provides an immunochromatographic device for determining the presence or absence of an analyte in a sample solution from which the analyte should be extracted, comprising:
(a) a prefilter pad made of a porous material comprising a sample deposition zone and an analyte extraction zone wherein one or more mobile extraction solutions are impregnated;
(b) a conjugate pad made of a porous material comprising a conjugate zone wherein a mobile solution comprising a labeled analyte-specific binding agent is impregnated;
(c) a detection membrane made of a porous material and comprising an analyte detection zone wherein an analyte-specific capturing agent and a control capturing agent are immobilized; and
(d) an absorbent material comprising a waste collection zone, capable of absorbing the excess of liquid from the sample solution; and optionally
(e) a plastic material to support and/or cover components (a) to (d) of the device;
   this device being characterized in that one or more of the extraction solutions comprises an animal serum or plasma component.

In some particular embodiments of the immunochromatographic device of the invention, the analyte is a Streptococcal antigen, and in specific cases a Group A Streptococcal antigen (Strep A) and/or a Group B Streptococcal antigen (Strep B).

In some particular embodiments of the immunochromatographic device of the invention, the sample is a swab sample, such as a throat swab sample or a bacteriological loop sample.

In some particular embodiments of the immunochromatographic device of the invention, one of the extraction solutions comprises a nitrite salt and an animal serum or plasma component. In some other particular embodiments, one of the extraction solutions comprises an acid and an animal serum or plasma component. In still other embodiments, a first extraction solution comprises an acid and a second extraction solution comprises a nitrite salt; and either the first extraction solution, the second extraction solution, or both extraction solutions comprise an animal serum or plasma component.

In some particular embodiments of the immunochromatographic device of the invention, the acid is selected from the group consisting of acetic acid, citric acid, sulfuric acid, hydrochloric acid, nitric acid, phosphoric acid and weak organic acids. The acid concentration in the extraction solution is in a range selected from about 0.05 M to about 0.6 M, about 0.2 M to about 0.5 M, or about 0.35 M to about 0.45 M. In some particular embodiments, the nitrite salt is sodium nitrite. The extraction solution is in a range selected from about 0.5 M to about 6 M, about 2 M to about 5 M, or about 3.5 M to about 4.5 M. In some particular embodiments, the animal serum or plasma component is selected from the group consisting of fetal bovine serum (FBS), fetal calf serum (FCS) and donor goat serum (DGS). The concentration of animal serum or plasma component is in a range selected from about 5% (v/v) to about 20% (v/v), about 7.5% (v/v) to about 15% (v/v), or about 9% (v/v) to about 11% (v/v).

In a particular embodiment of the immunochromatographic device of the invention, the labeled analyte-specific binding agent is a gold-labeled anti-Strep A mono- or polyclonal antibody; the analyte capturing agent is an anti-Strep A mono- or polyclonal antibody; and the control capturing agent is a mono- or polyclonal antibody raised against the species of the host animal in which the gold-labeled anti-Strep A antibody.

In another particular embodiment of the immunochromatographic device of the invention, the labeled analyte-specific binding agent is a gold-labeled anti-Strep B mono- or polyclonal antibody; the analyte capturing agent is an anti-Strep B monoclonal or polyclonal antibody; and the control capturing agent is a mono- or polyclonal antibody raised against the species of the host animal in which the gold-labeled anti-Strep B antibody was raised.

### BRIEF DESCRIPTION OF THE DRAWINGS

FIG. 1A schematically shows an expanded perspective view of the immunochromatographic elements of one specific embodiment of the immunochromatographic device of the invention;

FIG. 1B schematically shows a perspective view of one specific embodiment of the immunochromatographic device of the invention, when all the immunochromatographic elements have been assembled;

FIG. 2 schematically shows a lateral view of one specific embodiment of the immunochromatographic device of the invention; some parts of the immunochromatographic device are magnified in order to better appreciate how the different immunochromatographic elements have been assembled

FIG. 3 is a picture showing one specific embodiment (strip tests) of the immunochromatographic device of the invention for the detection of Group A Streptococcus antigen, tested in the presence of: A) 10⁸ cells of Group A Streptococcus (positive); or B) 10⁸ cells of Group C Streptococcus (negative);

FIG. 4 is a picture showing another specific embodiment (simple cassette) of the immunochromatographic device of the invention for the detection of Group A Streptococcus antigen, tested in the presence of: A) 10⁸ cells of Group C Streptococcus (negative); B) and C) 10⁸ cells of Group A Streptococcus (positive);

FIG. 5A is a picture showing a further specific embodiment (StrepAID assay) of the immunochromatographic device of the invention for the detection of Group A Streptococcus antigen, tested in the presence of: A) and B) 10⁶ cells of Group A Streptococcus (positive); C) 10⁸ cells of Group A Streptococcus (positive);

FIG. 5B is a picture showing the same embodiment of immunochromatographic device as shown in FIG. 5A, an tested in the presence of clinical samples coming from Group A Streptococcus positive patients.

### DETAILED DESCRIPTION

The present invention relates to a rapid immunoassay for detecting specific analytes in biological samples and methods for using the same. The methods of the present invention do not require any extensive preparation steps and can be performed by individuals without specific training in laboratory techniques.

In a first aspect, the present invention provides a method for determining the presence or absence of an analyte in a sample with minimal sample preparation. The method of this invention comprises the following steps:
(a) providing a sample for which the presence or absence of an analyte should be determined;
(b) diluting said sample in a water-based solution to obtain a sample solution;
(c) loading a volume of the sample solution onto the sample deposition zone of an immunoassay device without further addition of reagents to said sample solution;
(d) extracting the analyte of interest from the sample solution by flowing the sample solution through an extraction zone present in the immunochromatographic device, wherein one or more mobile extraction solutions comprising extracting reagents have been impregnated in the extraction zone, and wherein at least one of these extraction solutions comprises an animal serum or plasma component;
(e) forming a labeled analyte-binding agent complex with a labeled analyte-specific binding agent;
(f) determining the presence or absence of said analyte in the sample by the presence or absence of a signal formed by the binding of said labeled analyte-binding agent complex to a capture agent specific for said analyte or said complex; and
(g) determining the presence of a positive control signal.

In the method of this invention, the extraction of the analyte is performed within the immunochromatographic device by enabling the sample solution to flow through the extraction zone of the device. The order of addition of the extraction reagents to the sample solution may vary and has no consequence on the results of the assay. Moreover, neutralization of the solution further to the extraction of the analyte from the sample solution is not necessary. The positive control signal acts as an internal control and indicates that the reagents are functional and the assay has been performed properly.

In particular embodiments, the present invention can be used for detecting the presence or absence of a Streptococcal antigen, especially Group A or Group B Streptococcus antigens, in samples requiring extraction of the antigen.

In one embodiment of the method of this invention, a sample solution that should be tested for the presence or absence of Streptococcal antigens is obtained by dipping and vigorously mixing a throat swab sample in a water-based solution comprising an acid. In some specific embodiments, the acid is selected from the group consisting of acetic acid, citric acid, sulfuric acid, hydrochloric acid, nitric acid, phosphoric acid and weak organic acids. The sample solution is then brought into contact with an immunochromatographic device, wherein a solution comprising a nitrite salt (e.g. sodium nitrite) and an animal serum has been impregnated in the sample receiving zone or in a distinct extraction zone in direct flow connection with the sample receiving zone. Streptococcal antigens are extracted within the immunochromatographic device with no further intervention of the user, as the sample solution flows through the extraction zone and is brought into contact with the extraction reagents. Following the extraction, the solution flows through the conjugate zone, the analyte detection zone and the waste collection zone which absorbs the excess liquid of the sample. Visual signals indicate whether the Streptococcal antigens have been detected and whether the test has been performed properly.

In another embodiment of the method of this invention, a sample solution that should be tested for the presence or absence of Streptococcal antigens is obtained by dipping and vigorously mixing a throat swab sample in a water-based solution comprising a nitrite salt (e.g. sodium nitrite). The sample solution is then brought into contact with an immunochromatographic device, wherein a solution comprising an acid and an animal serum has been impregnated in the sample receiving zone or in a distinct extraction zone in direct flow connection with the sample receiving zone. In some specific embodiments, the acid is selected from the group consisting of acetic acid, citric acid, sulfuric acid, hydrochloric acid, nitric acid, phosphoric acid and weak organic acids. Streptococcal antigens are extracted within the immunochromatographic device with no further intervention of the user, as the sample solution flows through the extraction zone and is brought into contact with the extraction reagents. Following the extraction, the solution flows through the conjugate zone, the analyte detection zone and the waste collection zone which absorbs the excess liquid of the sample. Visual signals indicate whether the Streptococcal antigens have been detected and whether the test has been performed properly.

In still another embodiment, a sample solution that should be tested for the presence or absence of Streptococcal antigens is obtained by dipping and vigorously mixing a throat swab in a water-based solution. The sample solution is then brought into contact with an immunochromatographic device, wherein 1) a first extraction solution comprising sodium nitrite and 2) a second extraction solution comprising an acid have been impregnated in the sample receiving zone or in a distinct extraction zone in direct flow connection with the sample receiving zone. In this case, the first extraction solution, the second extraction, or both extraction solutions comprise an animal serum or plasma component. In some specific embodiments, the acid of the second extraction solution is selected from the group consisting of acetic acid, citric acid, sulfuric acid, hydrochloric acid, nitric acid, phosphoric acid and weak organic acids. The two solutions described above may be impregnated in the immunochromatographic device in any particular sequence, as the order in which the sample solution encounters the extraction reagents will not affect the assay results. Streptococcal antigens are then extracted within the immunochromatographic device with no further intervention of the user, as the sample solution flows through the extraction zone and is brought into contact with the extraction reagents comprised in the two immobilized solutions. Following the extraction, the solution flows through the conjugate zone, the analyte detection zone and the waste collection zone which absorbs the excess liquid of the sample. Visual signals indicate whether the StrepA antigen has been detected and whether the test has been performed properly.

In some embodiments, the nitrite salt is sodium nitrite. However, various kind of nitrite salt may be used in the present invention and one skilled in the art is aware of commercially available sources suitable for the use described in the present invention. The nitrite solution comprises a concentration of nitrite salt of from about 0.5 M to about 6 M, preferably from about 2 M to about 5 M, and more preferably from about 3.5 M to about 4.5 M. The concentration of animal serum or plasma component in the extraction solution(s) is from about 5% (v/v) to about 20% (v/v), preferably from about 7.5% (v/v) to about 15% (v/v), and more preferably from about 9% (v/v) to about 11% (v/v). The acid solution comprises a concentration of acid of from about 0.05 M to about 0.6 M, preferably from about 0.2 M to about 0.5 M, and more preferably from about 0.35 M to about 0.45 M. In some embodiments, the extraction solution(s) comprising the extraction reagents are impregnated in the extraction zone of the immunochromatographic device and further heat-dried or lyophilized.

Besides the nitrite salt, the nitrite solution may also contain surfactants (e.g. Tween 20), other type of salts, and preservative agents (e.g. Sodium azide).

While in some embodiments of the invention the acid solution comprises acetic acid, a variety of other acids may be reacted with the nitrite salt to form the nitrous reagent. In some embodiments, the acid is selected from the group consisting of acetic acid, citric acid, sulfuric acid, hydrochloric acid, nitric acid, phosphoric acid and weak organic acids. The acid solution may also contain surfactants (e.g. Tween 20), salts, and preservative agents (e.g. Sodium azide).

A second aspect of the present invention provides an immunochromatographic device for the detection of the presence or absence of an analyte in a sample, wherein the extraction of the analyte is performed within the immunochromatographic device, thereby simplifying the extraction step and decreasing the possibility of user mistake.

Any immunochromatographic device, and in particular any lateral flow immunoassay device can be used for the assays in this invention, including test strip devices or devices having plastic housings, provided that either the sample receiving zone or a specific extraction zone following the sample receiving zone is impregnated with one or more extraction solutions comprising extraction reagents and an animal serum or plasma component. A suitable example of lateral flow immunoassay device that can be used in the present invention is described for instance in U.S. Pat. No. 7,241,417 which is incorporated herein by reference.

The immunochromatographic device of the invention is particularly suitable for detecting the presence or absence of Streptococcal antigens, especially Group A and/or Group B antigens, in samples requiring extraction of the antigen.

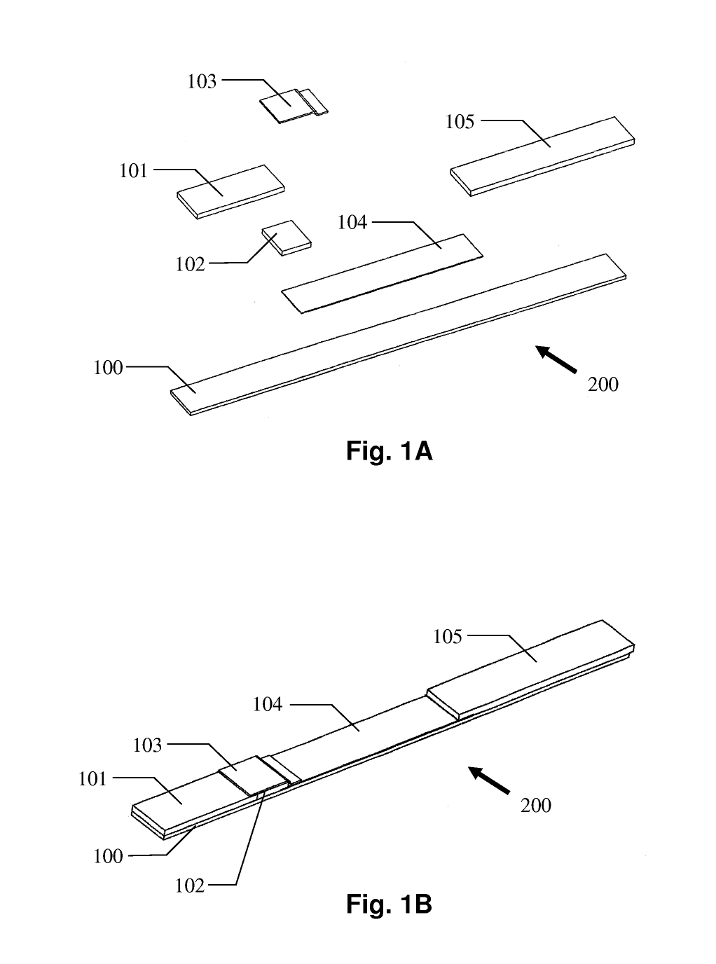

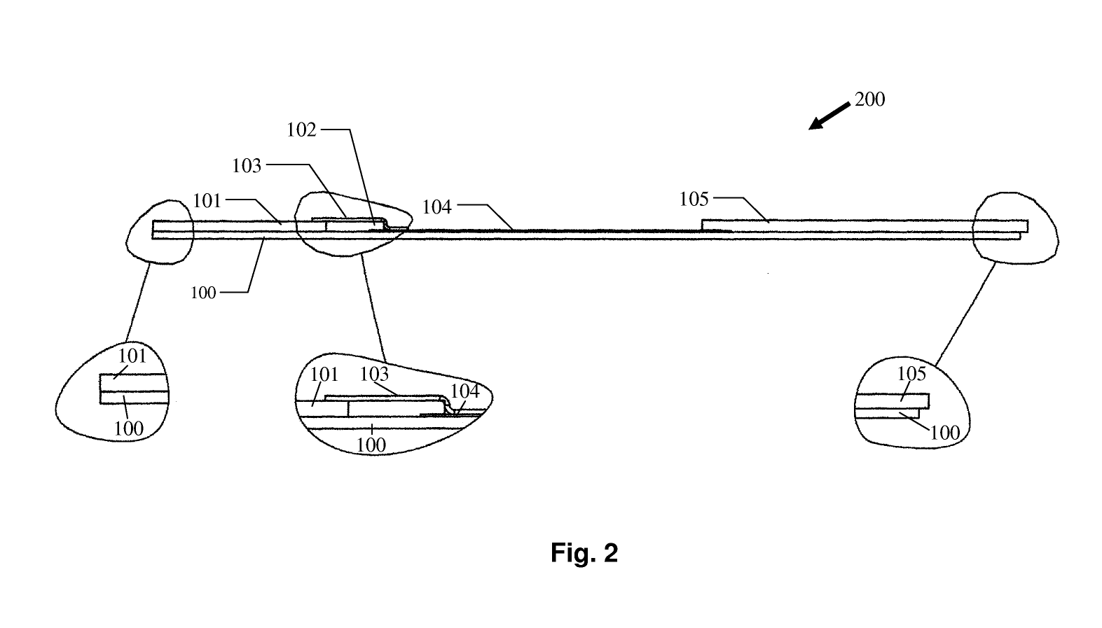

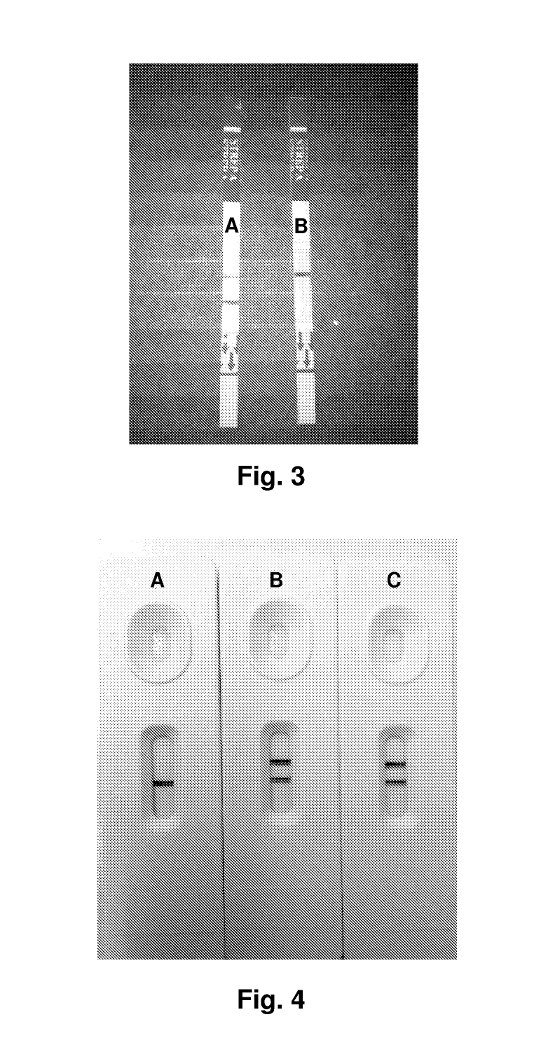

A specific embodiment of the immunochromatographic device 200 of the invention is a test strip as depicted in FIGS. 1A, 1B and 2. This immunochromatographic device comprises a prefilter pad 101, a conjugate pad 102, a “Clear Tape” (CT) adhesive film 103, a detection membrane 104, an absorbent material 105 and a backing element 100. The prefilter pad 101, the conjugate pad 102, the detection membrane 104 and the absorbent material 105 are all made of a porous material, allowing free passage of the liquid sample. A sample for which the presence or absence of an analyte should be determined is diluted in a water-based solution to obtain a sample solution. The sample solution is then loaded on a specific location of the prefilter pad 101 (sample receiving zone) of the immunochromatographic device 200. Once the sample solution has been impregnated in the prefilter pad 101, the sample solution flows successively through the analyte extraction zone located in the prefilter pad 101, the conjugate zone located in the conjugate pad 102, the analyte detection zone constituted by the detection membrane 104, and eventually reaches the waste collection zone constituted by an absorbent material 105 capable of absorbing the excess of liquid of the sample solution. In order to determine whether the sample contains the analyte of interest, the conjugate pad 102 comprises labeled analyte-specific binding agents which may form an analyte-binding agent complex with the analyte. Then, the formed complex flows through the analyte detection zone, wherein analyte-specific capturing agents and control capturing agents are immobilized at two distinct locations. The analyte specific capturing agents and control capturing agents are immobilized in the analyte detection zone following a specific pattern, for instance two distinct lines perpendicular to the direction of the flowing sample. A visual signal indicates whether the test has been performed properly (positive control) and whether the analyte of interest is present or absent (see FIGS. 3-5). Both the CT adhesive film 103 and the backing element 100, are used to maintain the other elements of the immunochromatographic device 200 in their respective position and do not carry out any tasks of chemical importance.

In one embodiment of the immunoassay device of this invention, the prefilter pad comprises a sample receiving zone wherein the sample solution is brought into contact with the device, and an extraction zone wherein the analyte is extracted. In another embodiment, the prefilter pad comprises a single deposition/extraction zone which combines the functions of the sample receiving zone and of the analyte extraction zone.

In some specific embodiments of the immunoassay device of the invention, the length of the prefilter pad is from about 9 mm to about 18 mm, the length of the conjugate pad is from about 3 mm to about 8 mm, the length of the detection membrane is from about 20 mm to about 30 mm, and the length of the adsorbent paper is from about 10 mm to about 25 mm. The length of the CT adhesive film is from about 5 mm to about 12 mm and the length of the backing element from about 40 mm to about 80 mm. The width of the above components is from about 2 to about 8 mm.

In some embodiments, the labeled analyte-specific binding agent present in the conjugate pad consists of a monoclonal or polyclonal antibody that specifically binds an antigen to be detected, and to which a substance or particle (such as gold, dye sols, colored latex) capable of producing a visually detectable signal is covalently or non-covalently bound. In one specific embodiment, the analyte-specific binding agent is a gold-conjugated polyclonal antibody directed to the carbohydrate antigen of Streptococcus Group A and/or Group B.

The term “sample” as used herein refers to any biological sample that could contain an analyte for detection which requires being extracted. In some embodiments, the sample is a swab sample (e.g. a throat swab sample), or a bacteriological loop containing bacteria (e.g. from culture on a media-containing plate, or a growth solution of bacteria).

As used herein, the term “minimal sample preparation” refers to the fact that the user of the present invention has only to dilute the sample to obtain a sample solution and load a volume of this sample solution on the immunoassay device of the invention in order to determine whether or not the antigen is present in the tested sample. No transfer from vial to vial is necessary and not more than one solution is employed in the sample preparation stage. For instance, a throat swab sample is diluted in a water-based solution comprising acetic acid to obtain a sample solution, and then a volume of this sample solution is loaded onto the sample deposition zone of a the immunoassay device.

“Diluting a sample” refers to mixing a solid sample or a non-homogeneous liquid sample with a water-based solution in a container as to obtain a “sample solution”, wherein biological microorganisms such as virus or bacteria are rendered more accessible to chemical and biological agents. By solid or non-homogeneous liquid sample is meant a sample which comprises a solid phase or a liquid sample which is adsorbed to a solid phase, such as a throat swab, or a bacteriological loop.

“Loading a volume of the sample solution” refers to transferring a specific volume of the sample solution from the container in which the sample has been diluted to the surface of the immunoassay device. This transfer may be accomplished via pipetting said volume from said container or by any other action causing a volume of the sample solution to be brought into contact with the immunoassay device.

To “extract” the antigen means to make accessible the binding site to which antigen specific binding agent will bind. This extraction may be, for instance, cleavage of the carbohydrate antigen from the cell wall of Group A and/or Group B Streptococcus, or disruption of cell walls or membranes to expose membrane-bound analytes or intracellular analytes. In the present invention, extraction of the antigen is always performed within the immunochromatographic device and not prior to the transfer of the sample solution to said device.

The term “analyte” refers to a compound or composition to be detected or measured in the test sample. The analyte has at least one binding site that may be recognized by a binding agent. The analyte of interest in an assay can be, for instance, a protein, a peptide, an amino acid, a nucleic acid, a hormone, a steroid, a vitamin, a pathogenic microorganism, a natural or synthetic chemical substance, a contaminant, or a drug. A more specific example of an analyte is Streptococcal antigens, especially Group A and/or Group B Streptococcal antigens.

As used herein, the term “sample receiving zone” or “receiving zone” means the portion of the assay device which receives the sample to be tested for the analyte in question. The liquid sample can then migrate, through lateral flow, from the sample receiving zone towards the waste collection zone. The sample receiving zone is often located at one edge of the immunoassay and is in flow contact with the analyte extraction zone. In some embodiments, the sample receiving zone and the analyte extraction zone are combined into a single receiving/extraction zone. The sample receiving zone is made of porous material.

As used herein, the term “analyte extraction zone” or “extraction zone” means the portion of the assay device in which the analyte is extracted from the sample solution. The analyte extraction zone is in flow contact with the sample deposition zone and the conjugate zone. In some embodiments, the sample receiving zone and the analyte extraction zone are combined into a single receiving/extraction zone. In the analyte extraction zone, one or more mobile extraction solutions comprising extracting reagents have been impregnated. At least one of the extraction solutions comprises an animal serum or plasma component. The analyte extraction zone is made of porous material.

The term “serum” or “animal serum” refers to a clear liquid obtained from an animal that separates from blood when it is allowed to clot completely. The serum used in the present invention includes, but is not limited to, Fetal Bovine Serum (FBS), Fetal Calf Serum (FCS) or Donor Goat Serum (DGS), but one skilled in the art is aware of other commercially available animal sera or plasma suitable for the use described in the present invention. In some embodiments of the immunoassay of the invention, the concentration of serum in the extraction solution(s) is from about 5% (v/v) to about 20% (v/v), preferably from about 7.5% (v/v) to about 15% (v/v), and more preferably from about 9% (v/v) to about 11% (v/v).

The term “plasma” or “animal plasma” refers to the straw-colored liquid component of an animal blood which contains dissolved proteins, glucose, clotting factors, mineral ions, hormones and carbon dioxide. Various kind of animal plasma may be used in the present invention and one skilled in the art is aware of commercially available source suitable for the use described in the present invention. In some embodiments of the immunoassay of the invention, the concentration of plasma in the extraction solution(s) is from about 5% (v/v) to about 20% (v/v), preferably from about 7.5% (v/v) to about 15% (v/v), and more preferably from about 9% (v/v) to about 11% (v/v).

The term “serum or plasma component” indicates either serum or plasma with all their natural constituents, or serum or plasma from which one or more constituents have been removed.

“Neutralization” of a solution refers to a reaction between an acidic solution and a basic solution to produce a “neutral” solution with a pH of about 7. The resulting pH may be not exactly neutral (pH=7). The pH may be for instance slightly basic to mimic the pH of a normal human serum (pH about 7.36-7.44). In the present invention, extraction of the analyte may be achieved by contacting a sample solution with an acidic solution. In the prior art, such an extraction is generally followed by the addition of a basic solution (e.g. Tris-sodium hydroxide) to the solution comprising the extracted analyte in order to obtain a neutral solution prior to running the immunoassay. In most cases, this is necessary to preserve the binding affinity of the antibodies to the antigen. However, in the present invention, no dedicated neutralization step is required.

As used herein, the term “porous material” refers to any material capable of providing lateral and or vertical flow. This would include material such as nitrocellulose, nitrocellulose blends with polyester or cellulose, untreated paper, porous paper, rayon, glass fiber, acrylonitrile copolymer or nylon. One skilled in the art will be aware of other porous materials that allow lateral or vertical flow. The term “lateral or vertical flow” refers to liquid flow in which all of the dissolved of dispersed components of the liquid are carried at substantially equal rates and with relatively unimpaired flow laterally or vertically through the material.

The term “mobile” as referred to herein means diffusively or non-diffusively attached, or impregnated. The reagents which are mobile are capable of dispersing with the liquid sample and carried by the liquid sample in the lateral flow. The term “immobile” as used herein refers to reagents which are attached to the support such that lateral flow of the liquid sample does not affect the placement of the immobile particle in the discrete zone of the porous material. Such attachment can be through covalent, ionic, or hydrophobic means. Those skilled in the art will be aware of means of attachment to immobilize various particles.

The term “labeled analyte-specific binding agent” as used herein refers to any particle, protein or molecule which specifically binds the analyte in question and has attached, conjugated or bound to it, either chemically, covalently or noncovalently, or ionicly or nonionicly any substance capable of producing a signal that is detectable by visual or instrumental means. Such labels producing a signal include chromogens, catalysts, fluorescent compounds, colloidal metallic and nonmetallic particles, dye particles, enzymes or substrates, organic polymers, latex particles, liposomes with signal producing substances and the like. The particle or molecule binding to the analyte can be natural or non-natural, preferably monoclonal or polyclonal antibody. In a specific embodiment of the invention, the labeled analyte-specific binding agent is a gold-labeled antibody directed to Group A and/or Group B Streptococcus carbohydrate antigen(s). Suitable antibody includes but is not limited to, monoclonal or polyclonal antibodies originating from mouse, rabbit or goat.

The term “analyte-specific capturing agent” as used herein refers to any particle or molecule which binds the analyte in question. The analyte-specific capturing agent is immobilized to the porous material of the analyte detection zone and is capable of forming a binding complex with the analyte of interest. The particle or molecule can be natural or synthetic, preferably monoclonal or polyclonal antibody. In a specific embodiment of the invention, the analyte-specific capturing agent is an antibody directed to Group A and/or Group B Streptococcus carbohydrate antigen(s). Suitable antibody includes but is not limited to, monoclonal or polyclonal antibodies originating from mouse, rabbit or goat.

The term “control capturing agent” as used herein refers to any particle or molecule which is capable of binding a control agent (such as a labeled BSA protein) or in some embodiments, any remaining labeled analyte-specific binding agent. The control capturing agent is immobilized to the porous material of the analyte detection zone. When the control reagent is bound to the immobilized control capturing agent, a visible positive control signal is emitted to indicate that the assay has been performed properly. The control capturing agent is applied to the porous material in any geometrical shape desired. For instance, the control capturing agent may be, without being limited to, a mono- or polyclonal antibody directed against the species of the host animal in which a gold labeled mono- or polyclonal antibody directed to Group A and/or Group B Streptococcus carbohydrate antigen(s) was raised.

The term “conjugate zone” as used herein refers to the portion of the assay device which is in flow contact with the porous material of the analyte extraction zone and the porous material of the analyte detection zone. In the conjugate zone, a mobile conjugate solution comprising the labeled analyte-specific binding agent and optionally a control agent is impregnated. When the sample solution flows through the conjugate zone, the labeled analyte-specific binding agent is binding the analyte and forming a complex analyte-binding agent, which moves onwards the analyte detection zone. The sample receiving zone is made of porous material.

The term “analyte detection zone” as used herein refers to the portion of the assay device which is in flow contact with the porous material of the conjugate zone and the porous material of waste collection zone. The analyte detection zone is comprised in the detection membrane which is made of a porous material, preferably nitrocellulose. The sample receiving zone, the conjugate zone, the analyte detection zone and the waste collection zone can be made of different material, or can be separate zones of the same porous member, for instance Fusion 5 from Whatman. The analyte detection zone contains the immobile analyte-specific capturing agent, the immobile control capturing agent, and optionally a control agent.

The term “waste collection zone” as used herein refers to the portion of the assay device which is in flow contact with the analyte detection zone. The sample solution migrates into the sample receiving zone of the immunoassay device, through the device to the opposite waste collection zone, which is capable of absorbing excess liquid sample.

The term “plastic material,” or “plastic cover,” or “cover”, or “cassette” as used herein refers to any plastic material which can support and/or cover the porous material of the device. Those skilled in the art will know of various materials that can be used for such purposes. The cover can be one continuous plastic or separate pieces. It must allow at least part of the analyte detection zone to be viewed, in particular at the locations wherein the control signal and the test signal (which reports the presence or absence of the analyte) could be seen. If the cover is not clear, then a window, gap or hole must be used so the results can be viewed. In addition, the cover must leave a portion of the sample receiving zone exposed so the sample can be applied to the receiving zone. Alternatively, the backing and plastic cover can be a molded plastic housing.

The following are examples of immunochromatographic assays of the present invention. These examples are offered by way of illustration and are not intended to limit the invention in any manner.

### Example 1

### StrepAID Lateral Flow Immunoassay for the Detection of Group A Streptococcal antigen

### General Overview

The StrepAID test is a rapid qualitative, lateral flow immunoassay for the detection of Streptococcus Group A carbohydrate (Strep A) antigen. The method employs polyclonal-dye conjugate and polyclonal solid phase antibodies to selectively identify streptococcus A with a high degree of sensitivity. As the sample solution flows through the absorbent device, the Strep A antigen is extracted from the sample solution and the labeled antibody-dye conjugate binds to the Strep A carbohydrate antigen forming an antibody-antigen complex. This complex binds to the anti-Strep A antibody in the test zone producing a purple color band. In the absence of Strep A there is no line in the test zone. The reaction mixture continues flowing through the absorbent device. Unbound conjugate binds to the reagents in the control zone producing a purple color band, indicating that proper volume of specimen has been added and membrane and the reagents are functioning correctly.

### Preparation of the Test Strip

### 1) Preparation of the Detection Membrane

Rabbit anti-Strep A polyclonal antibody (Fitzgerald #TF-70-XR83), which is used as analyte-specific capturing agent in the present assay, is diluted to a final concentration of about 1.5 mg/ml in a 20 mM sodium phosphate Na₂HPO₄ buffer solution at pH 7.4 (for 1 liter: 2.84 g Na₂HPO₄; adjusted to pH 7.4 with HCl) comprising 2.5% isopropanol. The control capturing agent control antibody, a polyclonal goat anti rabbit IgG (Fitzgerald #TF-41-GR30) is diluted to a final concentration of about 2 mg/ml in the same buffer solution. The solution comprising the Strep A capturing antibody and the solution comprising the goat anti rabbit IgG, are sprayed each one separately on a nitrocellulose membrane cast on a backing plastic material (card) (Millipore #HF13502S25). Typical dimension of the card is about 301 mm length, 60 mm width, and 166-205 μm thickness. Typically, the capturing solution and the control solution are sprayed as two thin lines of about 0.2-2 mm width, perpendicular to the flow of the sample solution. The card containing the nitrocellulose membrane is then impregnated with a membrane blocking solution (for 1 liter: 20 ml 0.5M NaP pH 7.4, 5 g BSA, 1 g Tween-20, 50 ml 10% PVP40 (Sigma PVP40), 20 g sucrose, 2.92 g NaCl; adjusted to pH 7.2-7.3 using 1M HCl).

### 2) Preparation of the Conjugate Pad

The labeled analyte-specific binding agent used in this specific assay is a gold-conjugated Rabbit anti-Strep A polyclonal antibody (Fitzgerald #TF-70-XR83). The gold-conjugated antibody is diluted to a final concentration of 3.5 OD (optical density units measured at 540 nm) in in a dilution vehicle at pH 7.3 containing 5% w/v sucrose, 1.25% w/v trehalose, 0.01M disodium tetraborate decahydrate, 1% v/v polyvinyl acetate, 0.2% w/v Tween 20, and 0.2% w/v Triton-×100. The total volume of solution should be about 11 μl for a typical conjugate pad of 0.2 cm² (Accuflow G glass fiber pad, Whatman #10534378). The conjugate pad is immersed in the conjugate solution and the homogeneous dispersion of the gold labeled antibody is then verified visually.

### 3) Preparation of the Prefilter Pad

A prefilter pad (Accuflow G glass fiber pad, Whatman #10534378) 12 mm-wide filter containing a solution of 4M Sodium Nitrite (Sigma #S2252) in 10% FBS (BioInd. #04-001-1a) is prepared for this test. The prefilter pad of typical dimension 12 mm×254 mm is immersed in 1.68 ml of a 4M Sodium Nitrite in 10% FBS solution. Optionally, the prefilter may be then vacuum- or heat-dried.

### 4) Test Strip Assembly

The test strips are prepared as illustrated in FIGS. 1A and 1B. The prefilter pad 101, the conjugate pad 102, the CT adhesive film 103 (Advanced Microdevices, India) and the absorbent material (Whatman #GB003) 105, are placed on the card comprising the detection membrane 104 and the backing element 100. The different components are placed as follows: the downstream end of the prefilter pad 101 is adjacent to the upstream end of the conjugate pad 102. The conjugate pad 102 overlaps the upstream end of the detection membrane 104 by 1-1.5 mm. The CT adhesive film 103 covers the entire conjugate-containing pad and overlaps the prefilter pad 101 by 1 mm and the detection membrane membrane 104 by 2 mm. The CT adhesive film 103 is made of polyester 25 μm thick, and one of its surfaces has glue on. Thickness of glue (excluding backing film) is 20 μm. The test strip is then introduced in a cassette as described in U.S. Pat. No. 7,241,417 to form the StrepAID immunoassay.

### Storage

### StrepAID is to be Stored at 2 to 25° C.

### Specimen Collection

The throat swab specimen is collected with the sterile swab provided in the kit. The swab is used to swab the posterior pharynx, tonsils and other inflamed areas. It is preferable to avoid touching the tongue, cheeks and teeth with the swab⁶. The testing is performed immediately after the specimens have been collected. Swab specimens may be stored in a clean, dry plastic tube for up to 8 hours at room temperature or 72 hours at 2-8° C. If a culture is desired, the swab tip can be gently rolled onto a Group A selective (GAS) blood agar plate before using the swab with the StrepAID.

### Assay Procedure

The lateral flow immunoassay device is placed on a flat horizontal surface and is held stable with one hand. The cover is carefully removed off the purple cap with the other hand. The purple cap contains a water-based solution comprising 0.4M acetic acid and 3% Tween 20 (v/v), and 0.01% Sodium azide (v/v). The sterile throat swab is then removed from the wrapper and is used to collect the throat swab specimen. The swab is rotated in the liquid placed in the purple cap for 20 sec and then pulled out carefully while squeezing it against the inner wall of the purple cap. The swab is discarded. The device is held stable with one hand and the purple cap is turned counterclockwise until it stops, and then turned back to the starting point. The procedure is then repeated two more times, the final position of the notches on the purple cap and the white base being aligned. The results may be read after 5 minutes. A purple line should appear at the control (C) mark. In case of positive results, an additional purple line should appear at the test (T) mark. In case of negative or unclear result read again after 5 more minutes.

### Quality Control

### 1) Internal Quality Control

Internal procedural controls are included in the test. A colored line appearing in the control line zone (C) is an internal positive procedural control. It confirms sufficient specimen volume, adequate membrane wicking and correct procedural technique (see FIGS. 3-5).

### 2) Cross-Reactivity

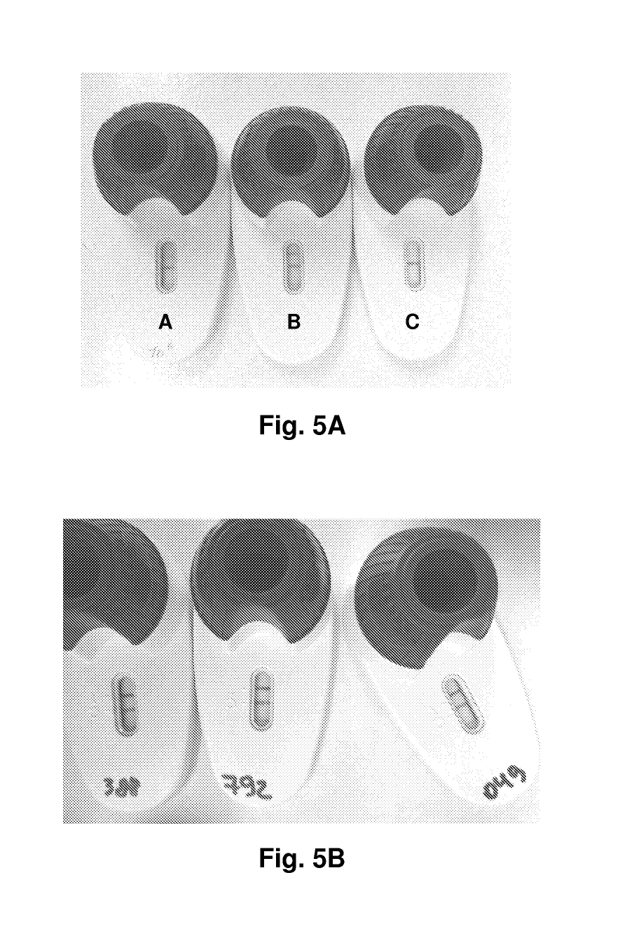

The following organisms were tested at 10⁶-10⁸ organisms per test and were all found to produce a negative result when tested with the StrepAID: Group B Streptococcus, Streptococcus pneumoniae, Streptococcus mutans, Staphylococcus aureus, Group C Streptococcus, Enterococcus faecalis, Aerococcus viridans, Staphylococcus saprofiticu, Staphylococcus epidermidis, Neisseria meningitides, Neisseria sicca, Klebsiella pneumoniae, Pseudomonas aeruginosa, Haemophilus influenzae, Candida albicans (see FIG. 5A). Samples very heavily inoculated with certain S. Aureus strains may produce a fade false positive result.

### 3) Intra-Batch Variability

TABLE 1

| Method | | S. pyogenes | | S. agalactiae | S. equi |
|---|---|---|---|---|---|
| | Results | Positive | Negative | Positive | Positive |
| StrepAID | Positive | 6 | 0 | 0 | 0 |
| | Negative | 0 | 6 | 6 | 6 |

### 4) Inter-Batch Variability

TABLE 2

| Method | | S. pyogenes | | S. agalactiae | S. equi |
|---|---|---|---|---|---|
| | Results | Positive | Negative | Positive | Positive |
| StrepAID | Positive | 3 | 0 | 0 | 0 |
| | Negative | 0 | 3 | 3 | 3 |

### BIBLIOGRAPHY

1. Appleton, R S, Victoria, B E, Tamer, D and Ayoub, E M. 1985. J. Lab. Clin. Med. 105:114-119.
2. Ayoub, E M and Wannamaker, L W. 1962. Pediatrics. 29:527-538.
3. Levinson, M L and Franck, P F. 1955. J. Bacteriol. 69:284-287.
4. Bisuo, A L and Ofek, I. 1974. J. Dis. Child. 127: 676-681.
5. Manual of Clinical Microbiology. 1980. 3^{rd} Edition.
6. Shea, Y. R., Specimen Collection and Transport, Clinical Microbiology Procedures Handbook, Isenberg, H. D., American Society of Microbiology, Washington D.C., 1.1.1-1.1.30, 1992.

1-41. (canceled)

42. A method for determining the presence or absence of an analyte in a sample with minimal sample preparation, said method comprising:
(a) providing a sample for which the presence or absence of an analyte should be determined and from which said analyte should be extracted;
(b) diluting said sample in a water-based solution to obtain a sample solution;
(c) transferring a volume of said sample solution to an immunochromatographic device comprising at least one porous element allowing the flowing of liquid solutions;
(d) extracting said analyte from said sample solution within said immunochromatographic device by contacting said sample solution with at least one mobile extraction solution impregnated in said device;
(e) forming a labeled analyte-binding agent complex by contacting the solution comprising the extracted analyte with a mobile solution comprising a labeled analyte-specific binding agent;
(f) determining the presence or absence of said analyte in the sample by the presence or absence of a signal formed by the binding of said labeled analyte-binding agent complex to an analyte capturing agent specific for said analyte or said labeled analyte-binding agent complex; and
(g) determining the presence of a positive control signal by the presence or absence of a signal formed by the binding of a control agent to a control capturing agent or the binding of the labeled analyte-specific binding agent of step (e) to a control capturing agent;
said method characterized in that at least one of the extraction solutions of step (d) comprises an animal serum or plasma component.

43. The method of claim 42, wherein the analyte is a Streptococcal antigen selected from a Group A Streptococcal antigen (Strep A), a Group B Streptococcal antigen (Strep B), and mixtures thereof.

44. The method of claim 42, wherein:
(i) step (b) consists in diluting the sample in a water-based solution comprising an acid; and wherein at least one of the extraction solutions of step (d) comprises a nitrite salt and an animal serum or plasma component; or
(ii) step (b) consists in diluting the sample in a water-based solution comprising a nitrite salt; and wherein at least one of the extraction solutions of step (d) comprises an acid and an animal serum or plasma component; or
(iii) step (b) consists in diluting the sample in a water-based solution; wherein a first extraction solution of step (d) comprises an acid and a second extraction solution of step (d) comprises a nitrite salt; and wherein the first extraction solution, the second extraction solution, or both comprise an animal serum or plasma component.

45. The method of claim 44, wherein the acid is selected from the group consisting of acetic acid, citric acid, sulfuric acid, hydrochloric acid, nitric acid, phosphoric acid and weak organic acids.

46. The method of claim 44, wherein the nitrite salt is sodium nitrite.

47. The method of claim 42, wherein the animal serum or plasma component is selected from the group consisting of fetal bovine serum (FBS), fetal calf serum (FCS), and donor goat serum (DGS).

48. The method of claim 42, wherein no neutralization step is required further to extraction step (d).

49. An immunochromatographic device for determining the presence or absence of an analyte in a sample from which said analyte should be extracted, said device comprising an analyte extraction zone wherein one or more mobile extraction solutions are impregnated, and wherein at least one of said extraction solutions comprises an animal serum or plasma component.

50. The immunochromatographic device of claim 49, wherein the analyte is a Streptococcal antigen selected from a Group A Streptococcal antigen, a Group B Streptococcal antigen, and mixtures thereof.

51. The immunochromatographic device of claim 49, wherein:
(i) one of the extraction solutions comprises a nitrite salt and an animal serum or plasma component;
(ii) one of the extraction solutions comprises an acid and an animal serum or plasma component; or
(iii) a first extraction solution comprises an acid and a second extraction solution comprises a nitrite salt; and wherein the first extraction solution, the second extraction solution, or both comprise an animal serum or plasma component.

52. The immunochromatographic device of claim 51, wherein the acid is selected from the group consisting of acetic acid, citric acid, sulfuric acid, hydrochloric acid, nitric acid, phosphoric acid and weak organic acids.

53. The immunochromatographic device of claim 51, wherein the nitrite salt is sodium nitrite.

54. The immunochromatographic device of claim 49, wherein the animal serum or plasma component is selected from the group consisting of fetal bovine serum (FBS), fetal calf serum (FCS) and donor goat serum (DGS).

55. An immunochromatographic device for determining the presence or absence of an analyte in a sample solution from which said analyte should be extracted, comprising:
(a) a prefilter pad made of a porous material comprising a sample deposition zone and an analyte extraction zone wherein one or more mobile extraction solutions are impregnated;
(b) a conjugate pad made of a porous material comprising a conjugate zone wherein a mobile solution comprising a labeled analyte-specific binding agent is impregnated;
(c) a detection membrane made of a porous material and comprising an analyte detection zone wherein an analyte-specific capturing agent and a control capturing agent are immobilized; and
(d) an absorbent material comprising a waste collection zone, capable of absorbing the excess of liquid from the sample solution; and optionally
(e) a plastic material to support and/or cover components (a) to (d) of the device;
said device being characterized in that one or more of said extraction solutions comprises an animal serum or plasma component.

56. The immunochromatographic device of claim 55, wherein the analyte is a Streptococcal antigen selected from a Group A Streptococcal antigen, a Group B Streptococcal antigen, and mixtures thereof.

57. The immunochromatographic device of claim 55, wherein:
(i) one of the extraction solutions comprises a nitrite salt and an animal serum or plasma component;
(ii) one of the extraction solutions comprises an acid and an animal serum or plasma component; or
(iii) a first extraction solution comprises an acid and a second extraction solution comprises a nitrite salt; and wherein the first extraction solution, the second extraction solution, or both comprise an animal serum or plasma component.

58. The immunochromatographic device of claim 57, wherein the acid is selected from the group consisting of acetic acid, citric acid, sulfuric acid, hydrochloric acid, nitric acid, phosphoric acid and weak organic acids.

59. The immunochromatographic device of claim 57, wherein the nitrite salt is sodium nitrite.

60. The immunochromatographic device of claim 55, wherein the animal serum or plasma component is selected from the group consisting of fetal bovine serum (FBS), fetal calf serum (FCS) and donor goat serum (DGS).
